(19) United States
(12) Patent Application Publication
(10) Pub. No.: US 2018/0169428 A1
(43) Pub. Date: Jun. 21, 2018

(54) SUPPORT DEVICE AND USER TERMINAL

(71) Applicant: Seiko Epson Corporation, Tokyo (JP)

(72) Inventors: Yasunori YOSHINO, Matsumoto (JP); Hiroaki HOSOMI, Minowa (JP)

(21) Appl. No.: 15/737,555

(22) PCT Filed: Nov. 15, 2016
(86) PCT No.: PCT/JP2016/083841

§ 371 Date: Dec. 18, 2017

(30) Foreign Application Priority Data
Nov. 24, 2015 (JP) 2015-228456

(51) Int. Cl. A61N 2/02

(52) U.S. Cl. CPC A61N 2/02; A61B 5/0476

(57) ABSTRACT

A support device includes: a data acquisition unit which acquires brain activity data of a user; a stimulation unit which gives an electromagnetic stimulus corresponding to the brain activity data, to a head of the user; and a brain wave storage unit which stores a change in the brain activity data. The support device is configured to give an electromagnetic stimulus which causes alpha waves of the brain activity data to change, when acquiring the brain activity data.

### BACKGROUND

### Technical Field

The present invention relates to a support device and a user terminal.

### Related Art

A device (head-worn strap) which is worn on the head of a user, stimulates the brain and thereby activates the brain to maintain good health is disclosed (see, for example, JP-A-2005-278733). According to JP-A-2005-278733, a magnet piece and an aluminum piece are attached to the head-worn strap and the activation of the brain of the user is achieved because the blood circulation in the brain can be facilitated by the effect of the magnet and the brain can be stimulated by the effect of the aluminum.

Incidentally, giving a stimulus suitable for the activity state of the brain of each user can be expected to not only maintain good health but also achieve improvement in the mental aspects of the user, for example, strengthen the user's willpower to accomplish something or the like. However, the head-worn strap disclosed in JP-A-2005-278733 has no configuration to detect the activity state of the brain of the user and is simply configured to give a uniform stimulus to each user. Thus, the problem to be solved by the invention of this application is to provide a support device that can give a stimulus suitable for the activity state of the brain of each user.

### SUMMARY

The invention is made in order to solve the foregoing problem and can be realized in the following forms or application examples.

### Application Example 1

A support device according to this application example includes: a data acquisition unit which acquires brain activity data of a user; a stimulation unit which gives an electromagnetic stimulus corresponding to the brain activity data, to a head of the user; and a data change storage unit which stores a change in the brain activity data. The support device is configured to give an electromagnetic stimulus which causes a predetermined parameter of the brain activity data to change, when acquiring the brain activity data.

With the configuration of the support device according to this application example, when the data acquisition unit acquires the brain activity data of the user, the stimulation unit gives an electromagnetic stimulus which causes a predetermined parameter of the brain activity data to change. Therefore, based on the change in the predetermined parameter, a stimulus suitable for the activity state of the brain of the user can be given. Also, since the data acquisition unit acquires the brain activity data of the user and a change in the brain activity data of the user is stored in the data change storage unit, changes in the brain activity including a predetermined parameter of each user can be stored in advance and utilized.

### Application Example 2

In the support device according to the above application example, it is preferable that a technique selected from a group including EEG, MEG, fMRI, fNIRS, SPECT, and ECoG is used for the acquisition of the brain activity data.

With this configuration, the brain activity data of the user can be acquired using one of brain imaging techniques including EEG, MEG, fMRI, fNIRS, SPECT, and ECoG.

### Application Example 3

In the support device according to the above application examples, it is preferable that the predetermined parameter is an intensity of alpha waves and that the electromagnetic stimulus which tends to cause the intensity of the alpha waves to increase is given to the user.

There is a theory that concentration is in an enhanced state when the intensity of alpha waves is high. Therefore, by giving the user an electromagnetic stimulus which causes the intensity of alpha waves to increase as the predetermined parameter, it is possible to achieve mental support such as enhancing the concentration of the user.

### Application Example 4

It is preferable that the support device according to the above application examples has a communication function and changes a condition for the electromagnetic stimulus in such a way as to facilitate a tendency of a change in the predetermined parameter, based on data stored and accumulated in an external server.

With this configuration, the acquired brain activity data of the user is stored and accumulated in the external server by the communication function. Therefore, the brain activity data of various users thus accumulated can be analyzed and utilized. Also, the condition for the electromagnetic stimulus can be changed in such a way as to facilitate the tendency of a change in the predetermined parameter, based on the result of the analysis. Therefore, a stimulus more suitable for the activity state of the brain of the user can be given.

### Application Example 5

A user terminal used for the support device according to the above application examples includes a living body contact unit which comes in contact with the head of the user. It is preferable that the user terminal is wearable by the user in the state where the living body contact unit is in contact with the head.

With this configuration, the user terminal used for the support device is a wearable in the state where the living body contact unit is in contact with the head of the user. Therefore, an electromagnetic stimulus can be given while the brain activity data is acquired in the state where the user is working or studying, wearing the user terminal.

### Application Example 6

It is preferable that the user terminal according to the above application example has a power generation unit which generates electric power and that the user terminal is configured to be able to operate on the electric power from the power generation unit.

With this configuration, the user terminal has the power generation unit and operates on the electric power from the power generation. Therefore, there is no need to worry about a flat battery and the need for external power supply can be eliminated.

### Application Example 7

A support device according to this application example includes: a data acquisition unit which acquires brain activity data of a user; a stimulation unit which gives an electromagnetic stimulus to a head of the user, with the brain activity data being fed back; and a data change storage unit which stores a change in the brain activity data. The support device is configured to give an electromagnetic stimulus which facilitates an autosuggestion of the user.

With the configuration of the support device according to this application example, the brain activity data of the user acquired by the data acquisition unit is fed back and the stimulation unit gives an electromagnetic stimulus to the head of the user. A change in the brain activity data is stored in the data change storage unit. Therefore, if a sense of expectation or a sense of security that giving an electromagnetic stimulus will generate alpha waves in the brain serves as an autosuggestion, a certain change can be expected to occur in the mental aspects of the user even if a change in the predetermined parameter such as alpha waves is not actually confirmed.

### BRIEF DESCRIPTION OF DRAWINGS

FIG. 1 is a block diagram showing a schematic configuration of a support device and a user terminal according to the present embodiment.

FIG. 2 is a view showing electrode placement positions based on the International 10-20 System.

FIG. 3 is a schematic view showing a user terminal according to Example 1.

FIG. 4 is a schematic view showing a user terminal according to Example 2.

FIG. 5 is a schematic view showing a user terminal according to Example 3.

FIG. 6 is a schematic view showing a user terminal according to Example 4.

FIG. 7 is a schematic view showing a user terminal according to Example 5.

FIG. 8 is a schematic view showing a user terminal according to Example 6.

FIG. 9 is a schematic view showing a user terminal according to Example 7.

### DETAILED DESCRIPTION

An object of an embodiment of the invention is to provide a support device and a user terminal which support improvement in the mental aspects of the user. The improvement in the mental aspects refers to increasing, for example, the willpower to put something into action, the willpower to concentrate and grapple with something, the willpower to continue and accomplish something, or the like. Increasing one's willpower can be expected to have effects such as concentrating and grappling with study or work, having good results, and improving productivity. Willpower varies among individuals. Some people have strong willpower and some people have weak willpower. Also, willpower wears off as it is used.

As a method for improving willpower, active action by a human, for example, brain activation training or multitasking in which multiple things are executed in parallel, is considered effective. Also, there is a theory that when brain waves are in an alpha-wave state, the person is in a mentally relaxed state, has his/her ability to concentrate or think improved, and can exhibit high abilities. It is thought that if brain waves can be turned into the alpha-wave state when the person is studying or working just as usual, without carrying out any particular training or the like, the person can increase his/her willpower in various situations.

Thus, the inventors propose a support device and a user terminal that are worn on the head of a user and thus detect the state of brain waves of the user, give a stimulus corresponding to the activity state of the brain, and help to increase willpower according to the individual differences each of user and the mental state at the time. Hereinafter, an embodiment of the invention will be described referring to the drawings.

### Configuration of Support Device

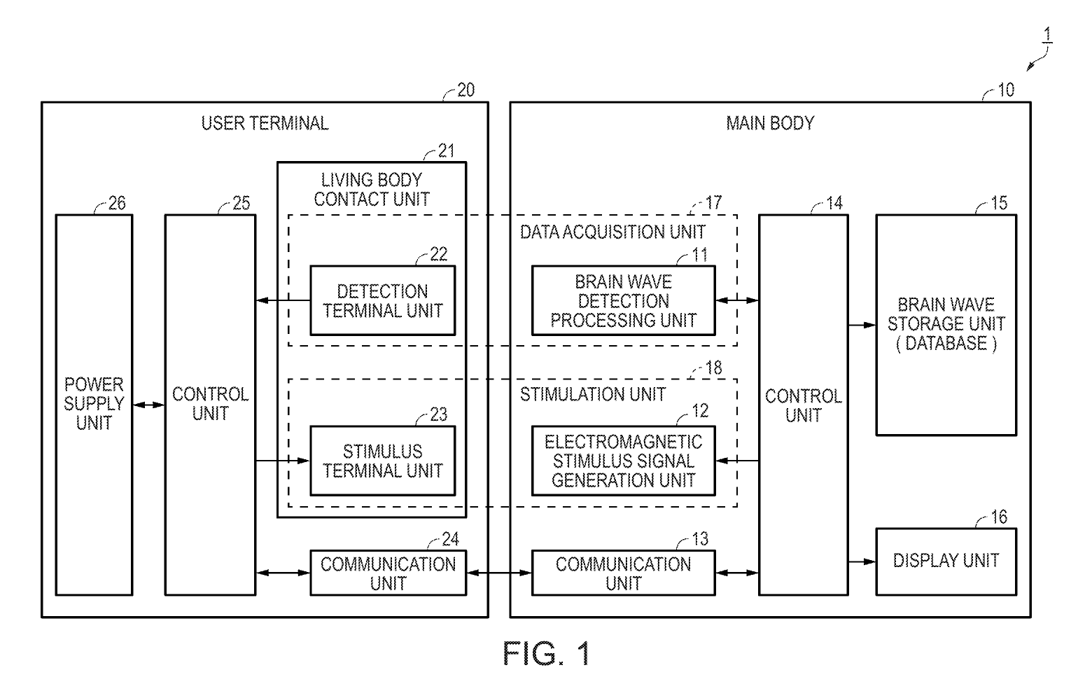

The configuration of the support device and the user terminal according to the embodiment will be described referring to FIG. 1. FIG. 1 is a block diagram showing a schematic configuration of the support device and the user terminal according to the embodiment.

As shown in FIG. 1, a support device 1 according to this embodiment has a main body 10 and a user terminal 20. The main body 10 has a brain wave detection processing unit 11, an electromagnetic stimulus signal generation unit 12, a communication unit 13, a control unit 14 which controls each part of the main body 10, a brain wave storage unit (database) 15 as a data change storage unit, and a display unit 16.

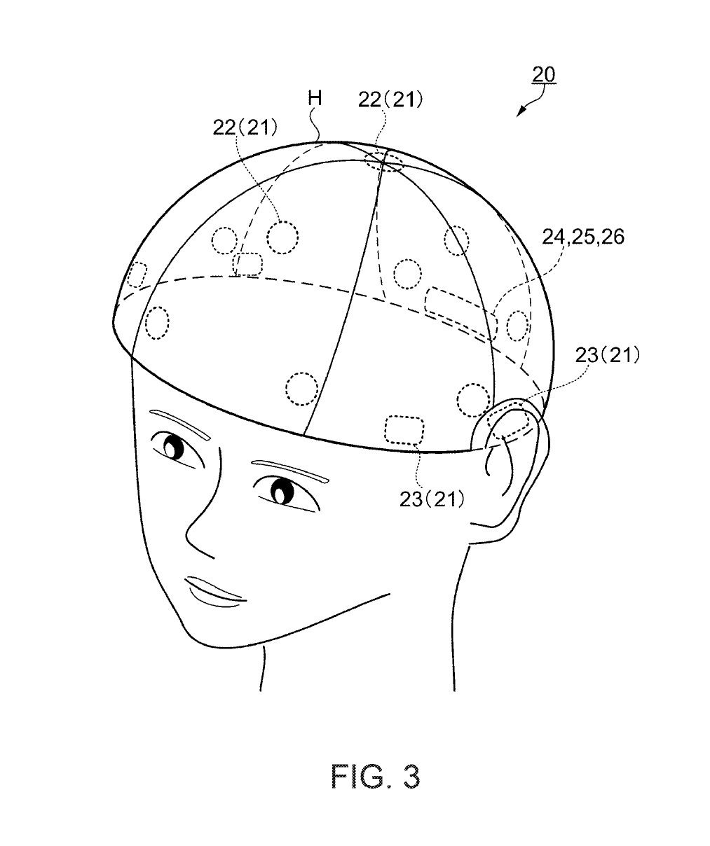

The user terminal 20 has a living body contact unit 21 including a detection terminal unit 22 and a stimulus terminal unit 23, a communication unit 24, a control unit 25 which controls each part of the user terminal 20, and a power supply unit 26 which supplies electric power to each part of the user terminal 20. The user terminal 20 is in a (wearable) form such that the user terminal 20 can be used in the state of being worn on a head H of a user (see FIG. 3). When the user wears the user terminal 20 on the head H, the living body contact unit 21 (detection terminal unit 22 and stimulus terminal unit 23) comes in contact with the head H of the user. Also, various forms of the user terminal 20 are conceivable and examples thereof will be described later.

The brain wave detection processing unit 11 and the detection terminal unit 22 form a data acquisition unit 17 which acquires brain activity data of the user. The data acquisition unit 17 (brain wave detection processing unit 11) is configured of a device adapted to one of techniques including EEG (electroencephalography), MEG (magnetoencephalography), fMRI (functional magnetic resonance imaging), fNIRS (functional near-infrared spectroscopy), SPECT (single photon emission computed tomography), and ECoG (electrocorticogram).

The support device 1 according to the embodiment is configured to detect brain waves as brain activity data. The relationship between human mental state and brain waves has been widely studied. By using brain waves as brain activity data, it is possible to utilize data obtained from those studies. Therefore, in the support device 1 according to the embodiment, it is preferable that the data acquisition unit 17 (brain wave detection processing unit 11) is configured of a device adapted to EEG for detecting brain waves, from among the above techniques.

The detection terminal unit 22 is configured of an electrode for detecting brain waves of the user. The brain waves of the user detected by the detection terminal unit 22 are sent to and processed by the brain wave detection processing unit 11 via communication between the communication unit 24 and the communication unit 13. The brain wave detection processing unit 11 acquires, for example, the intensity of alpha waves or the like, as a predetermined parameter of the brain waves. The control unit 14 causes the brain wave storage unit 15 to store the intensity data of the brain waves acquired by the brain wave detection processing unit 11. In the brain wave storage unit 15, a change in the intensity of the brain waves is stored.

The electromagnetic stimulus signal generation unit 12 and the stimulus terminal unit 23 form a stimulation unit 18 which gives an electromagnetic stimulus to the head H of the user. The electromagnetic stimulus is an electromagnetic stimulus that can be changed according to a change in the brain waves of the user. For example, a magnetism, weak electrical signal or the like can be used. If the electromagnetic stimulus is a magnetism, the stimulus terminal unit 23 is configured of an electromagnet that can change the intensity of magnetism, and the electromagnetic stimulus signal generation unit 12 generates an electrical signal corresponding to the intensity of magnetism generated by the stimulus terminal unit 23. If the electromagnetic stimulus is a weak electrical signal, the stimulus terminal unit 23 is configured of a positive electrode and a negative electrode that enable a weak current to flow, and the electromagnetic stimulus signal generation unit 12 generates a weak electrical signal applied between the positive electrode and the negative electrode of the stimulus terminal unit 23.

The embodiment is configured in such a way that, when the data acquisition unit 17 acquires brain waves of the user, the acquired brain wave data is fed back and the stimulation unit 18 gives the head H of the user an electromagnetic stimulus that causes the brain waves (alpha waves) to change. That is, the embodiment is configured in such a way that, while the data acquisition unit 17 acquires brain waves of the user, the stimulation unit 18 gives the head H of the user an electromagnetic stimulus corresponding to the state of brain waves of the user.

The control unit 14 has a CPU (central processing unit), a ROM (read only memory), a RAM (random access memory) or the like, not illustrated. The control unit 14 operates as each part of the control unit 14, for example, by developing a control program stored in the ROM into the RAM and causing the CPU to execute the control program developed in the RAM. Alternatively, instead of having the CPU, the control unit 14 may be configured of hardware such as an AISC (application specific IC) which realizes the same functions as the functions executed by the CPU and the control program, or may be configured of both the CPU and the ASIC.

The control unit 25, too, has a CPU (central processing unit), a ROM (read only memory), a RAM (random access memory) or the like, not illustrated, similarly to the control unit 14. The control unit 25 is installed in the user terminal 20 worn on the head H of the user. Therefore, it is preferable that the control unit 25 is smaller and lighter than the control unit 14. Thus, the control unit 25 may have a simpler configuration than the control unit 14.

The brain wave storage unit 15 continuously stores the intensity data of brain waves acquired by the brain wave detection processing unit 11 when the user is wearing the user terminal 20. Therefore, changes in the intensity of brain waves of the user are stored and accumulated in the brain wave storage unit 15. The brain wave storage unit 15 is configured of a non-volatile memory, for example, EEPROM (electrically erasable programmable read only memory) or the like, and stores information in a non-volatile and rewritable manner.

The display unit 16 displays the operating state of the support device 1 and data of the frequency, intensity and the like of brain waves acquired by the data acquisition unit 17. It is preferable that the display unit 16 can graphically display changes in the frequency and intensity of brain waves of the user, and that when the brain waves are in the alpha-wave state, the display unit 16 can clearly show that alpha waves are generated and the state of increase/decrease in alpha waves. As changes in the frequency and intensity of brain waves are displayed on the display unit 16, the user (or operator) can learn the state of brain waves (for example, whether alpha waves are generated, and an increase/decrease in alpha waves or the like).

The display unit 16 is configured of a display device such as a liquid crystal display device (LCD) or organic EL device (electroluminescent display). A touch panel may be provided in such a way as to overlap the display unit 16 and the operation of the support device 1 may be carried out via the touch panel.

The communication unit 13 and the communication unit 24 communicate with each other wirelessly or via wired connection. Thus, the acquisition of brain waves of the user and the provision of an electromagnetic stimulus to the head H of the user or the like can be carried out between the main body 10 and the user terminal 20. If the communication between the communication unit 13 and the communication unit 24 is carried out via wired connection, a communication cable for connecting to the main body 10 is needed. If the communication between the communication unit 13 and the communication unit 24 is carried out wirelessly, the user can move without being restrained by a communication cable.

The power supply unit 26 supplies electric power for causing each part of the user terminal 20 to operate. The power supply unit 26 is configured of a battery, rechargeable battery or the like. Also, the power supply unit 26 may be omitted and power may be supplied to the user terminal 20 from the main body 10 or an external power supply. If power is supplied to the user terminal 20 from the main body 10 or an external power supply, a power cable or the like for connecting to the main body 10 or the external power supply is needed. If the communication between the communication unit 13 and the communication unit 24 is carried out via wired connection and power is supplied from the main body 10 via a power cable, the power cable and the communication cable can be bundled together.

It should be noted that the main body 10 is not limited to a specific hardware configuration and can be arbitrarily realized by hardware and software. The main body 10 may be, for example, a personal computer or portable information terminal device, or may be an external device such as a cloud server in a cloud system. Also, the brain wave storage unit 15 may be provided in an external device such as a cloud server in a cloud system, separately from the main body 10. With such a configuration, various data accumulated in an external server at a laboratory, university or the like can be utilized via the internet or the like.

### Detection of Brain Waves and Stimulation by Support Device

As described above, the support device 1 according to the embodiment detects brain waves as brain activity data. Nerve cells responsible for information processing in the brain transmit information via electrical signals. Therefore, as nerve cells become active, a current is generated in the brain. This current generated in the brain, measured in the form of voltage, is brain waves. Bran waves include alpha waves (α waves) of approximately 8 Hz to 13 Hz, beta waves (β waves) of approximately 14 Hz to 30 Hz, theta waves (θ waves) of approximately 4 Hz to 7 Hz, and delta waves (δ waves) of approximately 0.5 Hz to 3 Hz.

It is said that a human is in a mentally relaxed state and has his/her ability to concentrate or think improved when the brain waves are in an alpha wave state of approximately 8 Hz to 13 Hz. It is said that a human has very high concentration and can exhibit high abilities particularly when the brain waves are in an alpha wave state of approximately 9 Hz to 11 Hz (mid-alpha waves). Therefore, in order to increase the willpower of the user, it is thought that the brain waves should be in the alpha wave state and that the state should be able to be maintained.

Thus, the support device 1 according to the embodiment defines a predetermined parameter of brain activity data as the intensity of alpha waves (in other words, the frequency and intensity of brain waves), and gives a stimulus suitable for the activity state of the brain (state of alpha waves) of the user. More specifically, the data acquisition unit 17 acquires the frequency and intensity of the brain waves of the user, and if the brain waves are not in the alpha wave state, an electromagnetic stimulus that causes alpha waves to be generated is given to the user. When the brain waves of the user turn into the alpha wave state, an electromagnetic stimulus that causes the intensity of the alpha waves to be increased further may be given to the user.

By using the support device 1 like this, the user can improve the willpower to put something into action, the willpower to concentrate and grapple with something, and the willpower to continue and accomplish something, and can consequently concentrate and grapple with his/her study or work, achieve good results, or improve productivity. It is also possible to improve the ability to make decisions and the ability to judge in the case where one needs to make a big decision in life such as employment tests or marriage proposal.

Here, alpha waves are generally considered to be measured from the occipital lobes of the brain and are also said to be measured from other parts than the occipital lobes. However, it is thought that, even with the same user, there are cases where the part from which alpha waves are measured may vary depending on the circumstance (environment) where the user is present and that there are also individual differences. Moreover, it is thought that the way of changing and the intensity of brain waves are different among individuals. Therefore, it is desirable to observe, in advance, at which part of the brain alpha waves tend to be measured and how brain waves (alpha waves) change with respect to a target user, and store the data thereof.

Meanwhile, it is also thought which part of the brain when given an electromagnetic stimulus and what condition that are likely to cause the generation of alpha waves are different among individuals. Therefore, it is desirable to observe, in advance, which part of the brain when given an electromagnetic stimulus and what condition (for example, frequency and output level) are likely to cause the generation of alpha waves, and what condition of the electromagnetic stimulus is likely to cause an increase in alpha waves, with respect to a target user, and store the data thereof.

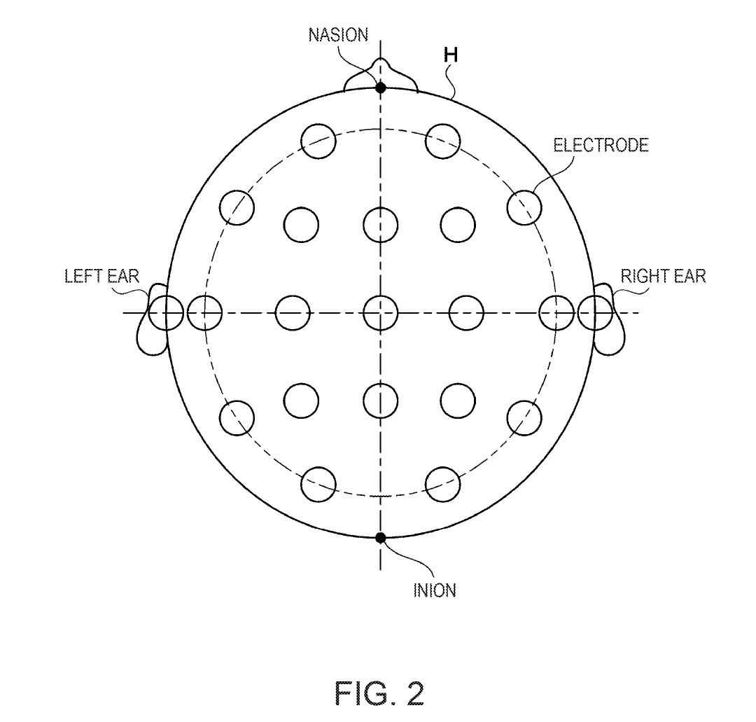

FIG. 2 is a view showing electrode placement positions based on the International 10-20 System. In FIG. 2, the head H of a human seen from above is illustrated with the forehead (nose side) facing upward. According to the International 10-20 System, the point of intersection between a line connecting the nasion and the inion (indicated by a dot-dash line) and a line connecting the left and right ears (indicated by a dot-dashed line) is defined as the center of the head H, and the head H is divided at equal intervals of 10% or 20%. Electrodes are placed, for example, at 21 positions.

When carrying out the above observation, the user terminal 20 having the detection terminal unit 22 placed at each position on the head H of the user, for example, based on the electrode placement positions according to the International 10-20 System shown in FIG. 2, is prepared. The user terminal 20 like this is mounted on the head H of the user, and the stimulus terminal unit 23 is placed in order at each position where the detection terminal unit 22 is placed. The state of brain waves when an electromagnetic stimulus is given by the electromagnetic stimulus signal generation unit 12 is acquired by the brain wave detection processing unit 11 via the detection terminal unit 22 placed at each position. The relationship between the electromagnetic stimulus and the state of generation of alpha waves at each position is observed. The stimulus terminal units 23 may be prepared in the same number as the detection terminal units 22 and placed at each position and an electromagnetic stimulus may be given at each position, switching the stimulus terminal units 23 in turn.

If such an observation is carried out in advance, which part of the brain of the user when given an electromagnetic stimulus and what condition (frequency and output level) that are likely to cause the generation of alpha waves at which part of the brain of the user can be grasped. Also, the placement of the detection terminal unit 22 and the stimulus terminal unit 23 on the user terminal 20 which the user wears on a regular basis can be properly set, based on the result of the observation. On the user terminal 20 which the user wears on a regular basis, the detection terminal unit 22 and the stimulus terminal unit 23 may be placed respectively at a position where the generation and detection of alpha waves can be effectively carried out on the head H of the user, and need not necessarily be placed at all the electrode placement positions based on the International 10-20 System.

Also, presenting the result of the observation like this to the user in advance is considered to be effective for the support device 1 to cause effective generation of alpha waves in the brain of the user (that is, improve the willpower of the user). For example, the above user terminal 20 is mounted on the head H of the user, and the stimulus terminal unit 23 is placed and an electromagnetic stimulus is given at a position where alpha waves are likely to be generated. Then, the state where alpha waves are generated is graphically displayed on the display unit 16 so as to get the user to see the state.

The user sees the state of generation of alpha waves displayed on the display unit 16 and can recognize which part of his/her brain, when given an electromagnetic stimulus, is likely to cause the generation of alpha waves. Then, by wearing the user terminal 20 on which the stimulus terminal unit 23 is placed at the position which is likely to cause the generation of alpha waves, the user obtains a sense of expectation or a sense of security that alpha waves are generated in the brain if an electromagnetic stimulus is given. Such a sense of expectation or sense of security can be expected to serve as an autosuggestion and work positively on the mental aspects of the user when the user uses the support device 1 on a regular basis.

When the user wears the user terminal 20 and uses the support device 1 on a regular basis, it is preferable that the device is programmed in such a way that the data acquisition unit 17 acquires brain waves of the user at a predetermined interval and that the simulation unit 18 automatically gives an electromagnetic stimulus that is likely to cause the generation of alpha waves or an electromagnetic stimulus that causes a tendency to increase alpha waves, if alpha waves are not generated or if the level of alpha waves is lowered. With this setting, when alpha waves are not generated in the brain of the user, the generation of alpha waves is prompted by the support device 1, and when alpha waves are generated, that state is maintained. Therefore, while wearing the user terminal 20, the user can increase the ability to concentrate and maintain the state of the high ability to concentrate.

Also, a sense of expectation or a sense of security that the support device 1 prompts the generation of alpha waves if the user wears the user terminal 20 and that when alpha waves are generated, the user is supported to maintain that state, works positively on the mental aspects of the user. Therefore, it can be expected that the user comes to act more actively and consequently achieve better results or improve productivity more.

If the results of observations of the various users and data or the like acquired in the state of regular use are accumulated in the brain wave storage unit 15 provided in an external device such as a cloud server in a cloud system, it is possible to carry out statistical analysis and utilize the result of the analysis. It is also possible to set a condition (frequency and output level or the like) of an electromagnetic stimulus given by the stimulation unit 18 in such a way as to facilitate a tendency of change in alpha waves, or effectively set the placement of the detection terminal unit 22 and the stimulus terminal unit 23 on the user terminal 20, based on the accumulated data.

### Examples of User Terminal

As the user terminal worn on the head H of the user, various forms can be considered due to the difference in shape or the like. Examples thereof will be described below. In the examples below, the basic configuration of each part is the same as the user terminal 20 shown in FIG. 1. However, the base part (base material) is different and therefore the placement position of each part and the number of units placed are different.

### Example 1

FIG. 3 is a schematic view showing a user terminal according to Example 1. As shown in FIG. 3, a user terminal 20 according to Example 1 is in the form of a head cap. The user terminal 20 has a substantially hemispherical shape wearable on the head H of the user. FIG. 3 shows an example in which the detection terminal unit 22 of the living body contact unit 21 is placed at positions corresponding to the frontal lobes, temporal lobes, parietal lobes, and occipital lobes, with the stimulus terminal unit 23 of the living body contact unit 21 being placed on the temples and the ears, and with the communication unit 24, the control unit 25, and the power supply unit 26 being placed on the back of the head. However, the placement of each unit is not limited to this.

The head cap-type user terminal 20 shown in FIG. 3 covers the entirety of the head H of the user and therefore can be worn in a stable state. Also, the detection terminal units 22 and the stimulus terminal units 23 can be placed respectively at optimum positions on the head H of the user, and can also be placed at each of the electrode placement positions based on the International 10-20 System.

### Example 2

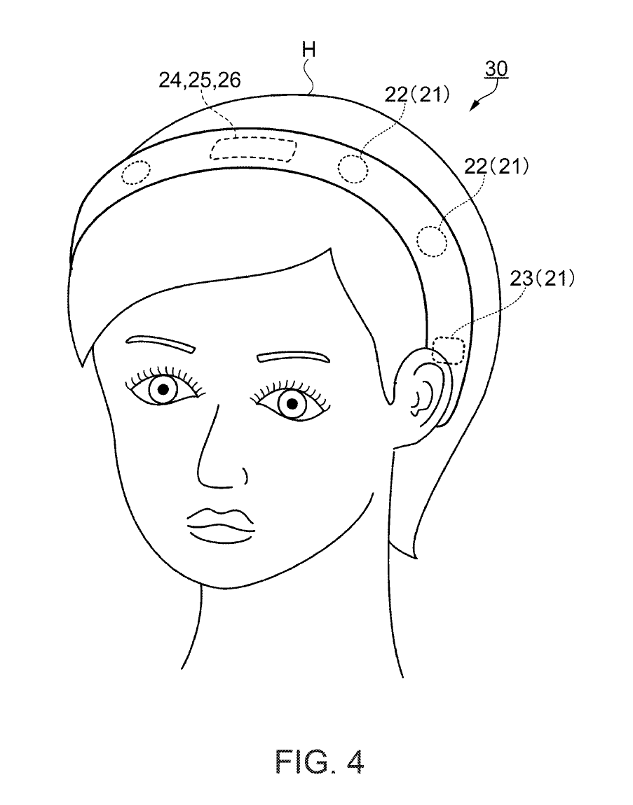

FIG. 4 is a schematic view showing a user terminal according to Example 2. As shown in FIG. 4, a user terminal 30 according to Example 2 is in the form of a hair accessory. The user terminal 30 is formed in a substantially C-shape with elasticity, for example, using plastics or the like as a base material, and is worn by being put over an area from the temporal regions (behind the ears) to the top of the head of the user. The detection terminal unit 22 and the stimulus terminal unit 23, the communication unit 24, the control unit 25, and the power supply unit 26 are placed on the side that comes in contact with the head H of the user along the substantially C-shape.

The hair accessory-type user terminal 30 shown in FIG. 4 looks like an accessory and therefore its appearance gives no inadequate impression even if the user terminal 30 is worn outdoors as well as indoors. However, since the user terminal 30 is not in a shape that covers the entirety of the head H of the user, the places where the detection terminal unit 22 and the stimulus terminal unit 23 are arranged are limited.

### Example 3

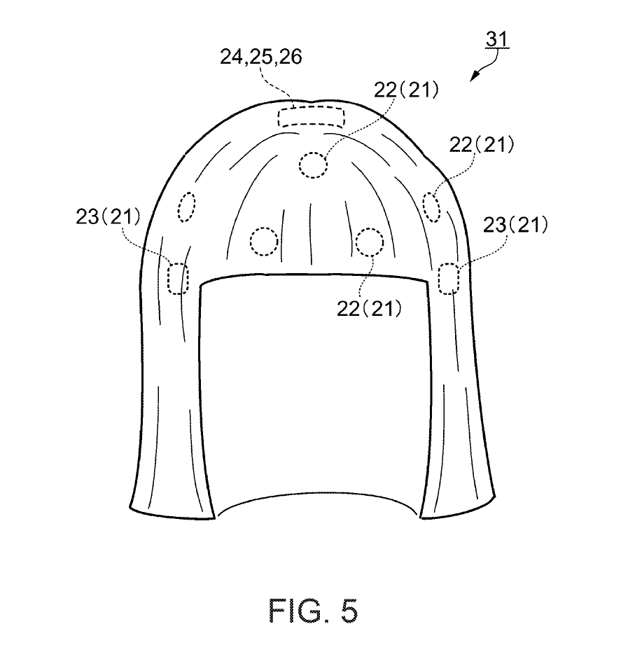

FIG. 5 is a schematic view showing a user terminal according to Example 3. As shown in FIG. 5, a user terminal 31 according to Example 3 is in the form of a wig. The detection terminal unit 22 and the stimulus terminal unit 23, the communication unit 24, the control unit 25, and the power supply unit 26 are placed on the inner side (side that comes in contact with the head H of the user) of the user terminal 31.

It can be said that the user terminal 31 is the head cap-type user terminal 20 according to Example 1 with hair arranged on the outside. Since the user terminal 31 covers the entirety of the head H of the user, as in Example 1, the detection terminal unit 22 and the stimulus terminal unit 23 can be placed respectively at optimum positions. Also, the user terminal 31 is in the form of a wig and therefore can be worn regardless of indoors or outdoors.

### Example 4

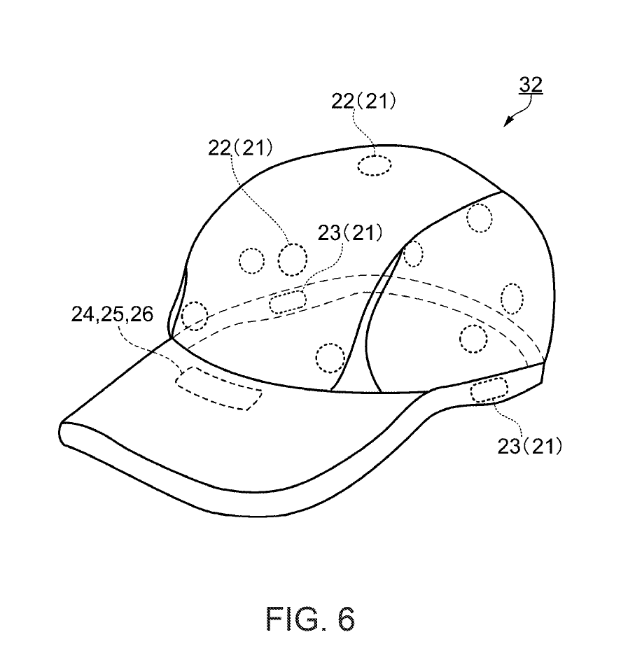

FIG. 6 is a schematic view showing a user terminal according to Example 4. As shown in FIG. 6, a user terminal 32 according to Example 4 is in the form of a cap. Since the user terminal 32, too, covers the entirety of the head H of the user, the detection terminal unit 22 and the stimulus terminal unit 23 can be placed respectively at optimum positions. Also, the communication unit 24, the control unit 25, and the power supply unit 26 can be placed on the visor part of the cap. The user terminal 32 is in the form of a cap and therefore suitable for the case of wearing it outdoors or the case of carrying out work or the like. Also, while the user terminal 32 shown in FIG. 6 is in the form of a cap with a visor on the front, the user terminal may be in the form of a hat with a brim on the circumference.

### Example 5

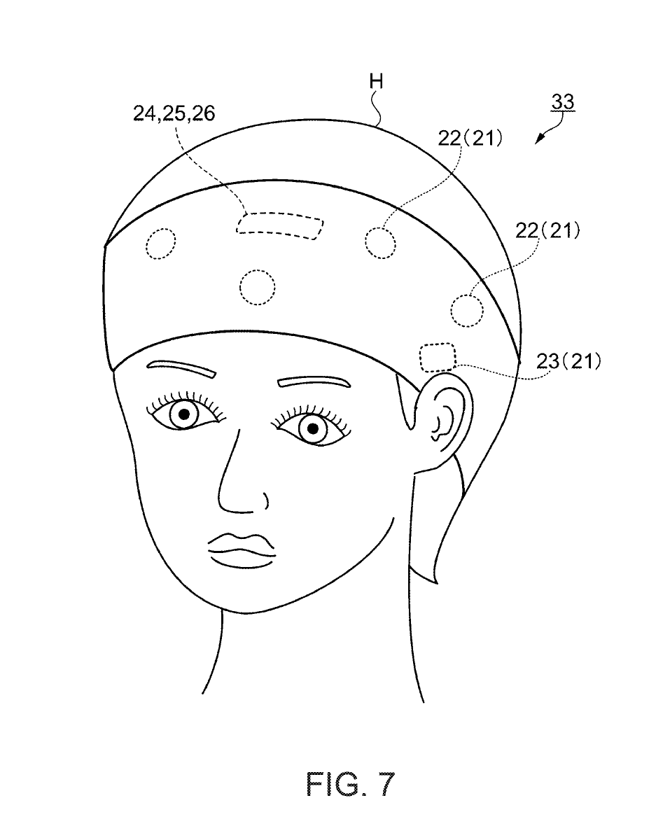

FIG. 7 is a schematic view showing a user terminal according to Example 5. As shown in FIG. 7, a user terminal 33 according to Example 5 is in the form of a hair band (headband). The user terminal 33 can be worn regardless of indoors or outdoors. Since the area covered by the user terminal 33 of the head H of the user can be greater than by the user terminal 30 according to Example 2, the limitation on the places where the detection terminal unit 22 and the stimulus terminal unit 23 are arranged can be reduced. Also, when wearing it, it is possible to finely adjust the positions of the detection terminal unit 22 and the stimulus terminal unit 23 with respect to the head H of the user.

### Example 6

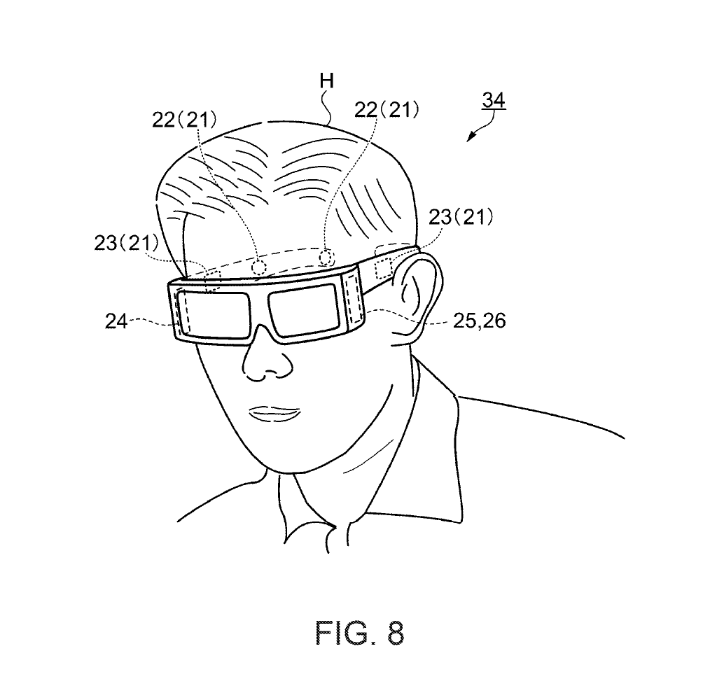

FIG. 8 is a schematic view showing a user terminal according to Example 6. As shown in FIG. 8, a user terminal 34 according to Example 6 is in the form of eyeglasses. In FIG. 8, the communication unit 24, the control unit 25, and the power supply unit 26 are placed near a hinge part of an eyeglasses frame, and the detection terminal unit 22 and the stimulus terminal unit 23 are placed at temple parts. The user terminal 34 is in the form of eyeglasses and therefore can be worn regardless of indoors or outdoors. However, the places where the detection terminal unit 22 and the stimulus terminal unit 23 are arranged are limited.

### Example 7

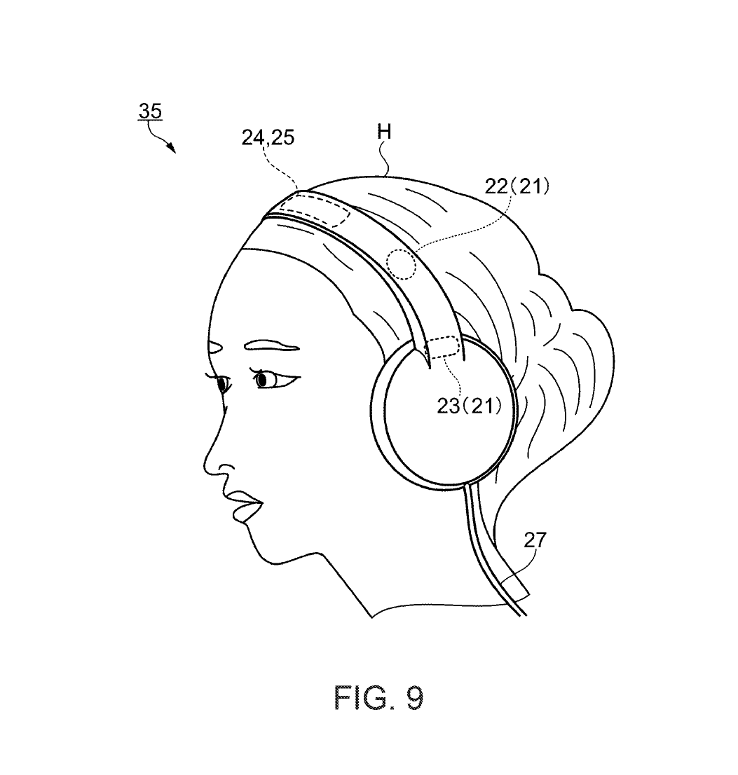

FIG. 9 is a schematic view showing a user terminal according to Example 7. As shown in FIG. 9, the user terminal 35 according to Example 7 is in the form of headphones. In FIG. 9, the stimulus terminal unit 23 is placed on a main body part including ear pad parts, which are parts to be butted against the ears of the headphones, and the detection terminal unit 22, the communication unit 24, and the control unit 25 are placed on a headset part supporting the main body at the top of the head. The main body part of the headphones is provided with a cable 27 for supplying music signals. Music signals may be supplied via the main body 10 from an external music signal generation device, or the main body 10 may include a music signal generation device.

Since the user terminal 35 is in the form of headphones and therefore can block external noises, the degree of concentration of the user can be increased further. Also, since the user terminal 35 can give the user a stimulus such as music that induces alpha waves from the headphones in addition to an electromagnetic stimulus from the stimulus terminal unit 23, the generation of alpha waves can be facilitated further. Also, since the headphones-type user terminal 35 can be supplied with electric power from the side of the main body 10 via the cable 27 for supplying music signals, the need for the power supply unit 26 can be eliminated.

The above embodiment simply illustrates one form of the invention and arbitrary modifications and applications can be made within the scope of the invention. As modifications, for example, the following is conceivable.

### Modification 1

In the embodiment, it is described that the power supply unit 26 of the user terminal 20 (30, 31, 32, 33, 34, 35) is configured of a battery or rechargeable battery. However, the invention is not limited to such a form. The power supply unit 26 may be configured of a power generation unit which generates electric power to allow each part of the user terminal 20 to operate. As the power generation unit, for example, a solar battery, or a mechanism (spring drive) which converts a mechanical power of a coil spring or the like into electrical energy, or the like may be employed. If the power supply unit (power generation unit) 26 generates and supplies electric power for allowing each part of the user terminal 20 to operate, there is no need to worry about a flat battery and the need for external power supply can be eliminated.

### Modification 2

In the embodiment, focusing on alpha waves of approximately 8 Hz to 13 Hz of brain waves, the configuration in which the data acquisition unit 17 acquires the intensity of alpha waves and in which the simulation unit 18 gives an electromagnetic stimulus that causes an increase in the intensity of alpha waves is employed. However, the invention is not limited to such a form. Focusing on brain waves of other frequencies than alpha waves as brain waves, a configuration in which an electromagnetic stimulus that causes an increase in the intensity of other brain waves than alpha waves may be employed.

For example, there is a theory that one's memory is enhanced when brain waves are in the state of theta waves of approximately 4 Hz to 7 Hz, and a theory that the state of theta waves is suitable for executing a passive behavior enforcement program such as overcoming dependence. Therefore, if one's objective is to enhance memory, diet, abstain from alcohol, quit smoking, or overcome drug dependence or the like, a configuration in which the data acquisition unit 17 acquires the intensity of theta waves and in which the simulation unit 18 gives an electromagnetic stimulus that causes an increase in the intensity of theta waves may be employed.

### Modification 3

In the embodiment, the data acquisition unit 17 acquires brain activity data of brain waves or the like. However, the invention is not limited to such a form. It is also possible to employ a configuration in which not only brain activity data but also biological information, for example, the amount of blood flow in the brain, electrocardiogram, eye movement, heart rate, pulse rate, perspiration, blood pressure, or body temperature is acquired and the result thereof is used. For example, it is said that if one's willpower drops due to accumulated stress, the heart rate is kept in a high state and the heartbeat interval is almost constant, whereas in a mentally relaxed state, the heart rate changes naturally. If not only the brain activity state (brain waves) of the user but also the biological information such as heart rate is acquired in this manner, it is possible to grasp the mental state of the user more accurately.

The invention of this application is described above on the assumption that a predetermined parameter of brain activity data of the user changes if an electromagnetic stimulus is given. However, this assumption is not necessarily essential. With the invention of this application, in the case where a sense of expectation or a sense of security that alpha waves will be generated if an electromagnetic stimulus is given serves as an autosuggestion, a certain change occurs in the mental state of the user even if no actual change is conformed in a predetermined parameter such as alpha waves by a data measuring device. Therefore, the invention of this application can be expected to have an effect.

The entire disclosure of Japanese Patent Application No. 2015-228456 filed on Nov. 24, 2015 is expressly incorporated by reference herein.

1. A support device comprising:
a data acquisition unit which acquires brain activity data of a user;
a stimulation unit which gives an electromagnetic stimulus corresponding to the brain activity data, to a head of the user; and
a data change storage unit which stores a change in the brain activity data,
the support device being configured to give an electromagnetic stimulus which causes a predetermined parameter of the brain activity data to change, when acquiring the brain activity data,
the support device comprising a communication function and changing a condition for the electromagnetic stimulus in such a way as to facilitate a tendency of a change in the predetermined parameter, based on data stored and accumulated in an external server.

2. The support device according to claim 1, wherein
a technique selected from a group including EEG, MEG, fMRI, fNIRS, SPECT, and ECoG is used for the acquisition of the brain activity data.

3. The support device according to claim 1, wherein
the predetermined parameter is an intensity of alpha waves, and the electromagnetic stimulus which tends to cause the intensity of the alpha waves to increase is given to the user.

4. (canceled)

5. A user terminal used for the support device according to claim 1,
the user terminal comprising a living body contact unit which comes in contact with the head of the user, the user terminal being wearable by the user in the state where the living body contact unit is in contact with the head.

6. The user terminal according to claim 5, comprising
a power generation unit which generates electric power,
the user terminal being configured to be able to operate on the electric power from the power generation unit.

7. A support device comprising:
a data acquisition unit which acquires brain activity data of a user;
a stimulation unit which gives an electromagnetic stimulus to a head of the user, with the brain activity data being fed back; and
a data change storage unit which stores a change in the brain activity data,
the support device being configured to give an electromagnetic stimulus which facilitates an autosuggestion of the user,
the support device comprising a communication function and changing a condition for the electromagnetic stimulus in such a way as to facilitate the autosuggestion, based on data stored and accumulated in an external server.

8. The support device according to claim 2, wherein
the predetermined parameter is an intensity of alpha waves, and the electromagnetic stimulus which tends to cause the intensity of the alpha waves to increase is given to the user.

9. A user terminal used for the support device according to claim 2,
the user terminal comprising a living body contact unit which comes in contact with the head of the user, the user terminal being wearable by the user in the state where the living body contact unit is in contact with the head.

10. A user terminal used for the support device according to claim 3,
the user terminal comprising a living body contact unit which comes in contact with the head of the user, the user terminal being wearable by the user in the state where the living body contact unit is in contact with the head.

11. A user terminal used for the support device according to claim 8,
the user terminal comprising a living body contact unit which comes in contact with the head of the user, the user terminal being wearable by the user in the state where the living body contact unit is in contact with the head.
